(19) United States
(12) Patent Application Publication
(10) Pub. No.: US 2004/0002519 A1
(43) Pub. Date: Jan. 1, 2004

(54) Combination of organic compounds

(76) Inventors: Robert Edson Damon, Randolph, NJ (US); Thomas Edward Hughes, Concord, MA (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/393,798

(22) Filed: Mar. 21, 2003

Related U.S. Application Data
(60) Provisional application No. 60/366,752, filed on Mar. 22, 2002.

(51) Int. Cl. A61K 31/4439; A61K 31/426; A61K 31/366; A61K 31/401; A61K 31/175; A61K 31/225

(52) U.S. Cl. 514/342; 514/369; 514/423; 514/460; 514/548; 514/592

(57) ABSTRACT

The present invention relates to a combination, especially a pharmaceutical composition, comprising as active ingredients

(i) a HMG-CoA reductase inhibitor or a or a pharmaceutically acceptable salt thereof;

(ii) (a) an insulin secretion enhancer or a pharmaceutically acceptable salt thereof or

(b) an insulin sensitizer or a pharmaceutically acceptable salt thereof; and,

in case of a pharmaceutical composition, a pharmaceutically acceptable carrier.

Combination of Organic Compounds

The present invention relates to a combination of at least two components selected from the group consisting of:

(i) a HMG CoA reductase inhibitor or a pharmaceutically acceptable salt thereof, selected from the group consisting of atorvastatin, cerivastatin, fluvastatin, lovastatin, pitavastatin, pravastatin, rosuvastatin, and simvastatin, and

(ii) a) an insulin secretion enhancer or a pharmaceutically acceptable salt thereof, or

b) an insulin sensitizer or a pharmaceutically acceptable salt thereof.

The invention also relates to a combination of at least two components selected from the group consisting of:

(i) a HMG CoA reductase inhibitor or a pharmaceutically acceptable salt thereof, and

(ii) a) an insulin secretion enhancer or a pharmaceutically acceptable salt thereof, selected from the group consisting of : tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, DPP-IV inhibitors, GLP1, GLP-1(7-36);Gln.sup.9-GLP-1(7-37); D -Gln.sup.9-GLP-1(7-37); acetyl-Lys.sup.9-GLP-1(7-37); Thr.sup.16-Lys.sup.18-GLP-1(7-37); and Lys.sup.18-GLP-1(7-37) or

b) an insulin sensitizer or a pharmaceutically acceptable salt thereof.

The invention furthermore relates to a method for the prevention, delay of progression or treatment of a disease and disorder which may be inhibited by the inhibition of HMG-Co-A reductase and/or by the enhancement of insulin secretion comprising administering to a warm-blooded animal, including man, in need thereof jointly therapeutically effective amounts of the composition comprising at least two components selected from the group consisting of:

(i) a HMG-CoA reductase inhibitor or a pharmaceutically acceptable salt thereof, selected from the group consisting of atorvastatin, cerivastatin, fluvastatin, lovastatin, pitavastatin, pravastatin, rosuvastatin, and simvastatin, and

(ii) a) an insulin secretion enhancer or a pharmaceutically acceptable salt thereof, or

b) an insulin sensitizer or a pharmaceutically acceptable salt thereof.

The invention furthermore also relates to a method for the prevention, delay of progression or treatment of a disease and disorder which may be inhibited by the inhibition of HMG-Co-A reductase and/or by the enhancement of insulin secretion comprising administering to a warm-blooded animal, including man, in need thereof jointly therapeutically effective amounts of the composition comprising at least two components selected from the group consisting of:

(i) a HMG-CoA reductase inhibitor or a pharmaceutically acceptable salt thereof, and

(ii) a) an insulin secretion enhancer or a pharmaceutically acceptable salt thereof, selected from the group consisting of: tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, DPP-IV inhibitors, GLP1, GLP-1(7-36);Gln.sup.9-GLP-1(7-37); D -Gln.sup.9-GLP-1(7-37); acetyl-Lys.sup.9-GLP-1(7-37); Thr.sup.16-Lys.sup.18-GLP-1(7-37); and Lys.sup.18-GLP-1(7-37) or

b) an insulin sensitizer or a pharmaceutically acceptable salt thereof.

Another embodiment of the invention relates to a combination according to the invention wherein the HMG CoA reductase inhibitor or a pharmaceutically acceptable salt thereof is selected from the group consisting of atorvastatin, fluvastatin, pitavastatin, and simvastatin.

Another preferred embodiment of the invention relates to a combination according to the invention wherein the HMG CoA reductase inhibitor or a pharmaceutically acceptable salt thereof is selected from the group consisting of fluvastatin, pitavastatin, and simvastatin.

Another more preferred embodiment of the invention relates to a combination according to the invention wherein the HMG CoA reductase inhibitor or a pharmaceutically acceptable salt thereof is selected from the group consisting of fluvastatin, pitavastatin.

The invention furthermore relates to a combination according to the invention wherein the insulin secretion enhancer or a pharmaceutically acceptable salt thereof is selected from the group consisting of sulfonylureas (SU), glinides, DPP-IV inhibitors, GLP1 and GLP1 agonists.

Another preferred embodiment of the invention relates to a combination according to the invention wherein the insulin secretion enhancer or a pharmaceutically acceptable salt thereof is selected from the group consisting of, tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, DPP-IV inhibitors, GLPI, GLP-1(7-36);Gln.sup.9-GLP-1(7-37); D -Gln.sup.9-GLP-1(7-37); acetyl-Lys.sup.9-GLP-1(7-37); Thr.sup.16-Lys.sup.18-GLP-1(7-37); and Lys.sup.18-GLP-1(7-37).

Another more preferred embodiment of the invention relates to a combination according to the invention wherein the insulin secretion enhancer or a pharmaceutically acceptable salt thereof is selected from the group consisting of, nateglinide and repaglinide.

Another more preferred embodiment of the invention relates to a combination according to the invention wherein the insulin secretion enhancer is nateglinide or a pharmaceutically acceptable salt thereof.

Another most preferred embodiment of the invention relates to a combination according to the invention wherein

a) the insulin secretion enhancer or a pharmaceutically acceptable salt thereof is nateglinide or a pharmaceutically acceptable salt thereof, or

b) the insulin secretion sensitizer is metformin.

Another more preferred embodiment of the invention relates to a combination according to the invention wherein the insulin secretion enhancer is

pyrrolidine, 1-[(3-hydroxy-1-adamantyl)amino]acetyl-2-cyano-, (S), or a pharmaceutically acceptable salt thereof.

Another most preferred embodiment of the invention relates to a combination according to the invention wherein the insulin secretion enhancer is 2-((5-cyanopyridin-2-yl)amino) ethyl or a pharmaceutically acceptable salt thereof.

Another most preferred embodiment of the invention relates to a combination according to the invention wherein the insulin secretion enhancer is ω-[(oxoquinazolinylalkoxy)phenyl]alkanoates and analogs thereof.

Another most preferred embodiment of the invention relates to a combination according to the invention wherein the insulin secretion enhancer is the compound 3-(4-(2-(2,3-Dihydro-1,4-benzothiazin-4-yl)ethoxy) phenyl)-2-ethoxypropanoic acid.

The invention furthermore also relates to a combination according to the invention wherein the combination is a pharmaceutical combination.

The invention furthermore also relates to a combination according to the invention for use in the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, hypothyroidism, survival post myocardial infarction (Ml), coronary heart diseases, hypertension in the elderly, familial dyslipidemic hypertension, and remodeling following hypertension.

The invention furthermore also relates to a combination according to the invention, for use in the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, hypothyroidism, survival post myocardial infarction (Ml), coronary heart diseases, hypertension in the elderly, familial dyslipidemic hypertension, and remodeling following hypertension.

The invention furthermore also relates to the use of a combination according to the invention for the manufacture of a medicament for the prevention, delay of progression or treatment of a disease and disorder which may be inhibited by the inhibition of HMG-CoA reductase and by the enhancement of insulin secretion.

Another embodiment of the invention relates to the use of a combination according to the invention for the manufacture of a medicament for the prevention, delay of progression or treatment of:

(α) a disease or condition selected from the group consisting of

hyperlipidaemia and dyslipidemia, atherosclerosis, insulin resistance and syndrome X, diabetes mellitus type 2, obesity, nephropathy, renal failure, e.g. chronic renal failure, hypothyroidism, survival post Ml, coronary heart diseases, hypertension in the elderly, familial dyslipidemic hypertension, and remodeling following hypertension (antiproliferative effect of the combination), all these diseases or conditions associated with or without hypertension; or

(β) endothelial dysfunction with or without hypertension; and

(γ) stroke, erectile dysfunction and vascular disease.

The present invention relates to the use of a combination according to the invention as described herein above before comprising as active ingredients

(i) a HMG-CoA reductase inhibitor or a pharmaceutically acceptable salt thereof;

(ii) (a) an insulin secretion enhancer or a pharmaceutically acceptable salt thereof or

(b) an insulin sensitizer or a pharmaceutically acceptable salt thereof;

for the manufacture of a medicament for the prevention, delay of progression or treatment of a disease and disorder which may be inhibited by the inhibition of HMG-CoA reductase and by the enhancement of insulin secretion, for example, for the prevention, delay of progression or treatment of hypertension, especially modest hypertension, congestive heart failure, endothelial dysfunction, impaired vascular compliance, IGT and type II diabetes mellitus.

Especially, the combination according to the present invention may be used, e.g., for the prevention, delay of progression or treatment of diseases and disorders selected from the group consisting of hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, hyperglycemia, hyperinsulinaemia, hyperlipidaemia, hypertryglyceridemia, insulin resistance, impaired glucose metabolism, conditions of impaired glucose tolerance (IGT), conditions of impaired fasting plasma glucose, obesity, diabetic retinopathy, macular degeneration, cataracts, diabetic nephropathy, glomerulosclerosis, diabetic neuropathy, erectile dysfunction, premenstrual syndrome, skin and connective tissue disorders, foot ulcerations and ulcerative colitis, endothelial dysfunction and impaired vascular compliance. Preferably, said combination may be used for the treatment of hypertension, especially ISH, congestive heart failure, endothelial dysfunction, impaired vascular compliance, IGT and type II diabetes mellitus.

HMG-CoA reductase inhibitors (also called β-hydroxy-β-methylglutaryl-co-enzyme-A reductase inhibitors) are understood, to be those active agents which may be used to lower the lipid levels including cholesterol in blood.

The class of HMG-CoA reductase inhibitors comprises compounds having differing structural features. For example, mention may be made of the compounds which are selected from the group consisting of atorvastatin, cerivastatin, fluvastatin, lovastatin, pitavastatin (formerly itavastatin), pravastatin, rosuvastatin, and simvastatin, or, in each case, a pharmaceutically acceptable salt thereof.

Preferred HMG-COA reductase inhibitors are those agents which have been marketed, most preferred is fluvastatin, atorvastatin, pitavastatin or simvastatin or a pharmaceutically acceptable salt thereof.

The term “antidiabetic” generally comprises the compounds, substances and compositions known to those of ordinary skill to be used in the treatment of type 1 and type 2 diabetes mellitus. This term in particular comprises insulin secretion enhancers and insulin sensitizers, as well as dipeptidyl peptidase IV (DPP IV) antagonists.

Insulin secretion enhancers are pharmacological active compounds having the property to promote secretion of insulin from pancreatic β-cells. Examples for insulin secretion enhancers include nateglinide, repaglinide, glucagon receptor antagonists, sulphonyl urea derivatives, incretin hormones, especially glucagon-like peptide-1 (GLP-1) or GLP-1 agonists, β-cell imidazoline receptor antagonists, and BTS 67582 described by T. Page et al in Br. J. Pharmacol. 1997, 122, 1464-1468.

Insulin secretion enhancers furthermore include short-acting insulin secretion enhancers, such as the new phenylalanine derivative nateglinide [N-(trans-4-isopropylcyclohexyl -carbonyl)-D-phenylalanine] (cf. EP 196222 and EP 526171) of the formula

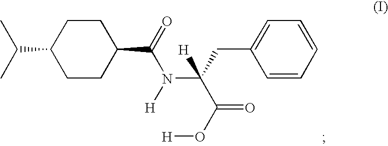

repaglinide [(S)-2-ethoxy-4-{2-[[3-methyl-1-[2-(1-piperidinyl)phenyl]butyl]amino]-2-oxoethyl}benzoic acid—cf. EP 589874]; calcium (2S)-2-benzyl-3-(cis-hexahydro-2-isoindolinlycarbonyl)-propionate dihydrate (mitiglinide—cf. EP 507534); furthermore representatives of the new generation of SUs such as glimepiride (cf. EP 31058); and in free or pharmaceutically acceptable salt form.

A preferred insulin secretion enhancer is repaglinide, most preferred is nateglinide. Repaglinde can be administered in the form as it is marketed e.g. under the trademark NovoNorm™.

The term nateglinide likewise comprises crystal modifications such as disclosed in EP 0526171 B1 or U.S. Pat. No. 5,488,510, respectively, the subject matter of which, especially with respect to the identification, manufacture and characterization of crystal modifications, is herewith incorporated by reference to this application, especially the subject matter of claims 8 to 10 (being directed to the H-form crystal modification) as well as the corresponding references to the B-form crystal modification.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The term “short-acting insulin secretion enhancer” comprises corresponding agents with a maximum secretion of insulin that is attained within one hour, preferably within 30 minutes, after the administration of the agent, most preferably within 20 minutes having a biological half-life, T ½, of less than two hours, preferably, 1.5 hours. The term long-acting insulin secretion enhancer” comprises corresponding agents with a maximum secretion of insulin that is attained more than one hour after administration of the agent.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these four references is herewith incorporated by reference in this specification.

The term “glucagon receptor antagonists” as used herein relates in particular to the compounds described in WO 98/04528, especially BAY27-9955, and those described in Bioorg Med. Chem. Left 1992, 2, 915-918, especially CP-99,711, J. Med. Chem. 1998, 41, 5150-5157, especially NNC 92-1687, and J. Biol Chem. 1999, 274; 8694-8697, especially L-168,049 and compounds disclosed in U.S. Pat. No. 5,880,139, WO 99/01423, U.S. Pat. No. 5,776,954, WO 98/22109, WO 98/22108, WO 98/21957 and WO 97/16442.

The sulphonyl urea (SU)derivative is, especially those which promote the secretion of insulin from pancreatic β-cells by transmitting signals of insulin secretion via SU receptors in the cell membrane, including (but are not limited to) tolbutamide; chlorpropamide; tolazamide; acetohexamide; 4-chloro-N-[(1-pyrolidinylamino)carbonyl]-benzensulfonamide (glycopyramide); glibenclamide (glyburide);glymepiride; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; and tolylcyclamide, or a pharmaceutically acceptable salt thereof.

Tolbutamide, glibenclamide, gliclazide, glibornuride, gliquidone, glisoxepid and glimepiride can be administered e.g. in the form as they are marketed under the trademarks RASTINON HOECHST™, AZUGLUCON™, DIAMICRON™, GLUBORID™, GLURENORM™, PRO-DIABAN™ and AMARYL™, respectively.

GLP-1 is a insulinotropic proteine which was described, e.g., by W. E. Schmidt et al. in Diabetologia 28, 1985, 704-707 and in U.S. Pat. No. 5,705,483. The term “GLP-1 agonists” used herein means variants and analogs of GLP-1 (7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826.

The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)0H, VAL⁸-GLP-1(7-37), GLY⁸-GLP-1(7-37), THR⁸-GLP-1(7-37), MET⁸-GLP-1 (7-37) and 4-imidazopropionyl-GLP-1. Special preference is also given to the GLP agonist analog exendin-4, described by Greig et al in Diabetologia 1999, 42, 45-50.

The term “β-cell imidazoline receptor antagonists” as used herein means compounds as those described in WO 00/78726 and by Wang et al in J. Pharmacol. Exp. Ther. 1996; 278; 82-89, e.g. PMS 812.

The term “insulin sensitizer” used herein means any and all pharmacological active compounds that enhance the tissue sensitivity towards insulin. Insulin sensitivity enhancers include, e.g., inhibitors of GSK-3, retinoid X receptor (RXR) agonists, agonists of Beta-3 AR, agonists of UCPs, antidiabetic thiazolidinediones (glitazones), non-glitazone type PPARy agonists, dual PPARy/PPARα agonists, antidiabetic vanadium containing compounds and biguanides, e.g., metformin.

The insulin sensitivity enhancer is preferably selected from the group consisting of antidiabetic thiazolidinediones, antidiabetic vanadium containing compounds and metformin.

Examples of “inhibitors of GSK-3” include, but are not limited to those disclosed in WO 00/21927 and WO 97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429,5,506,102, WO89/05355, WO91/06677, WO92/05447, WO93/11235, WO95/18380, PCT/US93/04399, PCT/US94/03795 and CA 2,034,220, which are incorporated by reference herein. It includes, but is not limited to, compounds that preferentially activate RXR over RAR (i.e. RXR specific agonists), and compounds that activate both RXR and RAR (i.e. pan agonists). It also includes compounds that activate RXR in a certain cellular context but not others (i.e. partial agonists). Compounds disclosed or described in the following articles, patents and patent applications which have RXR agonist activity are incorporated by reference herein: U.S. Pat. Nos. 5,399,586 and 5,466,861, WO96/05165, PCT/US95/16842, PCT/US95/116695, PCT/US93/10094, WO94/15901, PCT/US92/11214, WO93/11755, PCT/US93/10166, PCT/US93/10204, WO94/15902, PCT/US93/03944, WO93/21146, provisional applications 60,004,897 and 60,009,884, Boehm, et al. J. Med. Chem. 38(16):3146-3155, 1994, Boehm, et al. J. Med. Chem. 37(18):2930-2941, 1994, Antras et al., J. Biol. Chem. 266:1157-1161 (1991), Salazar-Olivo et al., Biochem. Biophys. Res. Commun. 204:157-263 (1994) and Safanova, Mol. Cell. Endocrin. 104:201-211 (1994). RXR specific agonists include, but are not limited to, LG 100268 (i.e. 2-[1-(3,5,5,8,8-pentamethyl-5,6,7,8-tetrahydro-2-naphthyl)-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzoic acid), and analogs, derivatives and pharmaceutically acceptable salts thereof. The structures and syntheses of LG 100268 and LG D 1069 are disclosed in Boehm, et al. J. Med. Chem. 38(16):3146-3155, 1994, incorporated by reference herein. Pan agonists include, but are not limited to, ALRT 1057 (i.e. 9-cis retinoic acid), and analogs, derivatives and pharmaceutically acceptable salts thereof.

Examples of “agonists of Beta-3 AR” include, but are not limited to CL-316,243 (Lederle Laboratories) and those disclosed in WO 99/29672, WO 98/32753, WO 98/20005, WO 98/09625, WO 97/46556, WO 97/37646 and U.S. Pat. No. 5,705,515.

The term “agonists of UCPs” used herein means agonists of UCP-1, preferably UCP-2 and even more preferably UCP-3. UCPs are disclosed in Vidal-Puig et al., Biochem. Biophys. Res. Commun., Vol. 235(1) pp. 79-82 (1997). Such agonists are a compound or composition which increases the activity of UCPs.

The antidiabetic thiazolidinedione (glitazone) is, for example, (S)-((3,4-dihydro-2-(phenyl -methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darg litazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl) -methyl]phenyl}methane (YM268), 5-{4-[2-(5-methyl-2-phenyl-4-oxazolyl)-2-hydroxyethoxy]-benzyl}-thiazolidine-2,4-dione (AD-5075), 5-[4-(1-phenyl-1-cyclopropanecarbonyl amino) -benzyl]-thiazolidine-2,4-dione (DN-108) 5-{[4-(2-(2,3-dihydroindol-1-yl)ethoxy)phenylmethyl}-thiazolidine-2,4-dione, 5-[3-(4-chloro-phenyll)-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl -benzyl)benzamide (KRP297).

More preferably, the thiazolidinedione is selected from the group consisting of 5-{[4-(2-(methyl-2-pyridinyl-amino)-ethoxy)phenyl]methyl}-thiazolidine-2,4-dione (rosiglitazone), 5-([4-(2-(5-ethyl-2-pyridyl)ethoxy)phenyl]-methyl}thiazolidine-2,4-dione (pioglitazone) and 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), MCC555, T-174 and KRP297, especially rosiglitazone, pioglitazone and troglitazone, or a pharmaceutically acceptable salt thereof.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-{[4-((3,4-dihydro-6-hydroxy-2,5,7,8-tetramethyl-2H-1-benzopyran-2-yl)methoxy)-phenyl]-methyl}thiazolidine-2,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]-methyl}-thiazolidine-2,4-dione (darglitazone, EP 0 332 332), 5-(2-naphthylsulfonyl)-thiazolidine-2,4-dione (AY-31637, U.S. Pat. No. 4,997,948), 5-{([4-(1-methyl-cyclohexyl)methoxy)-phenyl]methyl}-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{[4-(2-(2,3-dihydroindol-1-yl)ethoxy)phenyl]methyl}-thiazolidine-2,4-dione is described in B. B. Lohray et al., J. Med. Chem. 1998, 41, 1619-1630; Examples 2d and 3g on pages 1627 and 1628. The preparation of 5-[3-(4-chlorophenyl])-2-propynyl]-5-phenylsulfonyl) -thiazolidine-2,4-dione and the other compounds in which A is phenylethynyl mentioned herein can be carried out according to the methods described in J. Wrobel et al., J. Med. Chem. 1998, 41, 1084-1091.

In particular, MCC555 can be formulated as disclosed on page 49, lines 30 to 45, of EP 0 604 983 B 1; englitazone as disclosed from page 6, line 52, to page 7, line 6, or analogous to Examples 27 or 28 on page 24 of EP 0 207 605 B1; and darglitazone and 5-{4-[2-(5-methyl-2-phenyl-4-oxazolyl)-ethoxy)]benzyl}-thiazolidine-2,4-dione (BM-13.1246) can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

Non-glitazone type PPARy agonists are especially N-(2-benzoylphenyl)-L-tyrosine analogues, e.g. GI-262570, and JTT501.

The term “dual PPAR_{γ}/PPARα agonists” as used herein means compounds which are at the same time PPAR_{γ}and PPARα agonists. Preferred dual PPAR_{γ}/PPARα agonists are especially those ω-[(oxoquinazolinylalkoxy)phenyl]alkanoates and analogs thereof, or are very especially the compound 3-(4-(2-(2,3-Dihydro-1,4-benzothiazin-4-yl) ethoxy) phenyl)-2-ethoxypropanoic acid of formula (II)

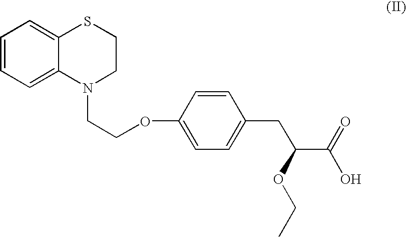

which is described in WO 99/20614, furthermore the compound NC-2100 ((±)-5-((7-benzyloxy-3-quinolyl) methyl)-2,4-thiazolidinedione) described by Fukui in Diabetes 2000, 49(5), 759-767.

Preferably, the antidiabetic vanadium containing compound is a physiologically tolerable vanadium complex of a bidentate monoprotic chelant, wherein said chelant is an α-hydroxypyrone or a-hydroxypyridinone, especially those disclosed in the Examples of U.S. Pat. No. 5,866,563, of which the working examples are hereby incorporated by reference, or a pharmaceutically acceptable salt thereof.

In a more preferred embodiment, the insulin sensitizer is metformin or a pharmaceutically acceptable salt thereof such as the mono-hydrochloride.

The preparation of metformin (dimethyldiguanide) and its hydrochloride salt is state of the art and was disclosed first by Emil A. Werner and James Bell, J. Chem. Soc. 121, 1922, 1790-1794. Mefformin, can be administered e.g. in the form as marketed under the trademark GLUCOPHAGE™. The metformin may be present in free form or in the form of a pharmaceutically acceptable salt and includes corresponding stereoisomers as well as the corresponding crystal modifications, e.g. solvates and polymorphs. Preferably, the metformin is metformin hydrochloride.

The term “dipeptidyl peptidase IV antagonists” or “DPP IV antagonists” comprises all activity reducing effectors of the enzyme dipeptidyl peptidase IV as defined and specifically named in WO 97/40832, e.g. isoleucyl-thiazolidid, and also the compounds of the following formulae

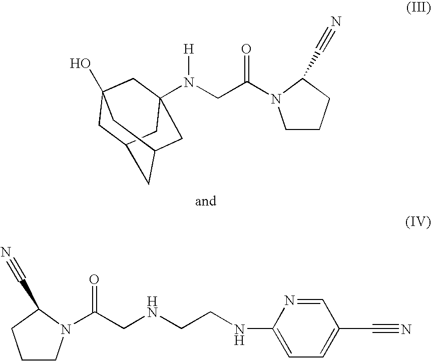

or a pharmaceutically acceptable salt of these compounds, in particular the dihydrochloride of compound of formula (IV). DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal. The DPP-IV inhibitor can be peptidic or, preferably, non-peptidic. The compound of formula (III) and its preparation is disclosed in WO 00/34241 whereas the compound of formula (IV), its dihydrochloride and its preparation is disclosed in WO 98/19998, the contents of which are hereby incorporated by reference. DPP-IV inhibitors are in each case generically and specifically disclosed e.g. in WO 98/19998, DE 196 16 486 Al, WO 00/34241, WO 95/15309, WO 01/47514 and WO01/52825 in each case in particular in the compound claims and the final products of the working examples, the subject-matter of the final products, the pharmaceutical preparations and the claims are hereby incorporated into the present application by reference to these publications. Preferred are compounds 1-{2-[(5-cyanopyridin-2-yl)amino]ethylamino}acetyl-2(S)-cyano-pyrrolidine dihydrochloride (cf. example 3 of WO98/19998), (S)1-[(3-hydroxy-1-adamantyl)amino]-acetyl-2-cyano-pyrrolidine (cf. example 1 of W00/34241) and pyrrolidine, 1-[(3-hydroxy-1-adamantyl)amino]acetyl-2-cyano-, (S) of formula

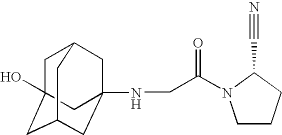

described in WO 01/47514 and WO01/52825.

The corresponding active ingredients or a pharmaceutically acceptable salts thereof may also be used in form of a solvate, such as a hydrate or including other solvents, used for crystallization.

Most preferred are dual combinations of one statin and one antidiabetic, but the combination of the present invention can also be a triple combination, e.g. of one statin and two antidiabetics.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

Preferably, the jointly therapeutically effective amounts of the active agents according to the combination of the present invention can be administered simultaneously or sequentially in any order, e.g. separately or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a HMG-CoA reductase inhibitor and insulin secretion enhancer and/or an insulin sensitizer, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes, e.g. less gain of weight. An additional and preferred aspect of the persent invenion is the prevention, delay of progression or treatment of the condition of isolated systolic hypertension and impaired vascular compliance which means decreased vascular elasticity.

In particular, all the more surprising is the experimental finding that the combination of the present invention results in a beneficial, especially a synergistic, therapeutic effect but also in benefits resulting from combined treatment such as a surprising prolongation of efficacy, a broader variety of therapeutic treatment and surprising beneficial effects on diseases and conditions as specified hereinbefore or hereinafter.

The pharmaceutical activities as effected by administration of representatives a HMG-CoA reductase inhibitor or an insulin secretion enhancer or (b) an insulin sensitizer, or of the combination of active agents used according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

The pharmaceutical activities as effected by administration of representatives of the class of HMG-COA reductase inhibitor or insulin secretion enhancers, respectively, or of the combination of active agents used according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

A “disease or condition which may be inhibited by the enhancement of insulin secretion” or a “disease or condition that may be inhibited by insulin sensitization” as defined in this application comprises, but is not limited to hyperglycemia, hyperinsulinaemia, hyperlipidaemia, hypertryglyceridemia, insulin resistance, impaired glucose metabolism, conditions of impaired glucose tolerance (IGT), conditions of impaired fasting plasma glucose, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance.

Furthermore, it has been found that the chronic co-administration of either an insulin sensitizer or an insulin secretion enhancer imparts the beneficial effect on blood vessel morphology and function and results in a decrease of vascular stiffness and correspondingly in a maintenance and in an improvement of vascular compliance.

Accordingly, it has been found that the addition of an insulin sensitizer and/or an insulin secretion enhancer to that of an HMG-CoA reductase inhibitor or a pharmaceutically acceptable salt thereof would potentiate the effect on systolic blood pressure and further improve vascular stiffness/compliance. The benefit of these combinations may also extend to an additional or potentiated effect on endothelial function, and improve vascular function and structure in various organs/tissues including the kidney, heart, eye and brain. Through the reduction in glucose levels, an anti-thrombotic and anti-atherosclerotic effect can also be demonstrated. Reduction of glucose would prevent or minimize the glycosylation of any structural or functional protein within the cardio-renal system.

All the more surprising is the experimental finding that the combined administration of a HMG-CoA reductase inhibitor and insulin secretion enhancer and/or an insulin sensitizer, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes, e.g. less gain of weight. The term “potentiation” shall mean an increase of a corresponding pharmacological activity or therapeutical effect, respectively. Potentiation of one component of the combination according to the present invention by co-administration of an other component according to the present invention means that an effect is being achieved that is greater than that achieved with one component alone.

The term “synergistic” shall mean that the drugs, when taken together, produce a total joint effect that is greater than the sum of the effects of each drug when taken alone.

Hypertension, in connection with a “disease or condition which may be inhibited by the inhibition of HMG-CoA reductase inhibitor”, a “disease or condition which may be inhibited by the enhancement of insulin secretion”, a “disease or condition that may be inhibited by insulin sensitization” includes and is not limited to mild, moderate and severe hypertension as defined in Journal of Hypertension 1999, 17:151-183, especially on page 162.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of modest hypertension or isolated systolic hypertension that is beneficial to all diabetic patients regardless of their hypertensive status, e.g. reducing the risk of negative cardiovascular events by two different modes of action.

The pharmaceutical composition according to the present invention as described hereinbefore and hereinafter may be used for simultaneous use or sequential use in any order, for separate use or as a fixed combination.

Under certain circumstances, drugs with different mechanisms of action may be combined. However, just considering any combination of drugs having different modes of action but acting in the similar field does not necessarily lead to combinations with advantageous effects.

The pharmaceutical composition according to the present invention comprises a “kit of parts” in the sense that the components can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components. Preferably, there is at least one beneficial effect, e.g. a mutual enhancing of the effect of

(i) a HMG-CoA reductase inhibitor or a pharmaceutically acceptable salt thereof;

(ii) (a) an insulin secretion enhancer or a pharmaceutically acceptable salt thereof or

(b) an insulin sensitizer or a pharmaceutically acceptable salt thereof;

in particular a potentiation or a synergism, e.g. a more than additive effect, additional advantageous effects, less side effects, a combined therapeutical effect in a non-effective dosage of one or each of the components, especially a potentiation or a strong synergism.

The invention furthermore relates to a commercial package comprising the combination according to the present invention together with instructions for simultaneous, separate or sequential use.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commerically available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

In case of HMG-CoA reductase inhibitors, preferred dosage unit forms of HMG-CoA reductase inhibitors are, for example, tablets or capsules comprising e.g. from about 5 mg to about 120 mg, preferably, when using fluvastatin, for example, 20 mg, 40 mg or 80 mg (equivalent to the free acid) of fluvastatin, for example, administered once a day.

The insulin secretion enhancer nateglinide (I) is preferably administered to the warm-blooded animal in a dosage in the range of about 5 to 1200, more preferably 25 to 800, mg/day, when the warm-blooded animal is a human of about 70 kg body weight. Preferred dosages contain 30 mg, 60 mg, 120 mg or 180 mg of nateglinide to be administered preferably before the main meals. In a low dose combination, the dosage of nateglinide to be administered preferably is 30 mg, 40 mg or furthermore 60 mg. Depending on the number of main meals the dose regimen are two times a day (BID) or three times a day (TID) or four times a day (QID).

The insulin secretion enhancer repaglinide is preferably administered in a dosage range of about 0.01 mg to about 8 mg, more preferred from about 0.5 to about 6 mg.

The insulin sensitizer metformin is preferably administered in a dosage range of about 100 mg to about 1200 mg per dose unit, especially 500 mg, 850 mg or 1000 mg. In a low dose combination, metformin is preferably administered in a dosage of 125 mg, 250 mg or 500 mg.

### EXAMPLE 12:

Hard gelatin capsule:

| Component | Amount per unit [mg] |
|---|---|
| Capsule | |
| Fluvastatin Sodium¹⁾ | 21.481²⁾ |
| Calcium Carbonate | 62.840 |
| Sodium Bicarbonate | 2.000 |
| Microcrystalline Cellulose | 57.220 |
| Pregelatinized Starch | 41.900 |
| Purified Water³⁾ | Q.S. |
| Magnesium Stearate | 1.050 |
| Talc | 9.430 |
| Target Capsule Fill Weight | 195.92 |
| Capsule Shell | |
| Hard gelatin Capsule Shell | 48.500 |
| Branding Ink (pre-printed) | |
| White Ink | Trace |
| Red Ink | Trace |
| Target Capsule Weight | 244.42 |

¹⁾includes a 2% overage for moisture
²⁾20 mg of free acid is equivalent to 21.06 mg Na salt
³⁾partially removed during processing

### EXAMPLE 13:

Hard gelatin capsule

| Component | Amount per unit [mg] |
|---|---|
| Fluvastatin Sodium | 42.962¹⁾²⁾ |
| Calcium Carbonate | 125.680 |
| Sodium Bicarbonate | 4.000 |
| Microcrystalline Cellulose | 114.440 |
| Pregelatinized Starch | 83.800 |
| Purified Water³⁾ | Q.S. |
| Magnesium Stearate | 2.100 |
| Talc | 18.860 |
| Target Capsule Fill Weight | 391.840 |
| Capsule Shell | |
| Hard gelatin Capsule Shell | 76.500 |
| Branding Ink (pre-printed) | |
| White Ink | Trace |
| Red Ink | Trace |
| Target Capsule Weight | 468.34 |

¹⁾includes a 2% overage for moisture
²⁾20 mg of free acid equivalent to 21.06 mg Na salt
³⁾partially removed during processing

### EXAMPLE 14:

Round, slightly bi-convex, film-coated tablets
with beleved edges:

| Component | Amount per unit [mg] |
|---|---|
| Table Core | |
| Fluvastatin Sodium¹⁾ | 84.24²⁾ |
| Cellulose Microcrystalline/Micro- | 111.27 |
| crystalline cellulose fine powder | |
| Hypromellose/Hydroxypropyl | 97.50 |
| methyl cellulose (Methocel | |
| K100LVP CR; HPMC100 cps) | |
| Hydroxypropyl cellulose (Klucel | 16.25 |
| HXF) | |
| Potassium hydrogen carbonate/ | 8.42 |
| Potassium bicarbonate | |
| Povidone | 4.88 |
| Magnesium stearate | 2.44 |
| Core Tablet Weight | 325.00 |
| Coating | |
| Coating premix - Opadry Yellow | 9.75 |
| (00F22737) | |
| Total Weight | 334.75 |
| Water, purified³⁾ | Q.S. |

¹⁾84.24 mg of the sodium salt of fluvastatin is equivalent to 80 mg of fluvastatin free acid
²⁾to be adjusted for moisture (LOD)
³⁾removed during processing

### EXAMPLE 12:

108,000 tablets, each which contain 120 mg of nateglinide are prepared as follows:

| | | | |
|---|---|---|---|
| Composition: | nateglinide | 12.960 | kg |
| | lactose, NF | 30.564 | kg |
| | microcrystalline cellulose, NF | 15.336 | kg |
| | povidone, USP | 2.592 | kg |
| | croscarmellose sodium, NF | 3.974 | kg |
| | colloidal silicon dioxide, NF | 1.382 | kg |
| | magnesium stearate, NF | 1.231 | kg |
| | coating: opadry yellow | 1.944 | kg |
| | purified water, USP* | Q.S. | |

*removed during process

Preparation process: The microcrystalline cellulose, povidone, part of the croscarmellose sodium, nateglinide and lactose are mixed in a high shear mixer and afterwards granulated using purified water. Alternatively, the microcrystalline cellulose, povidone, a portion of the croscarmellose sodium, nateglinide and lactose are granulated in a collette gral granulator with the addition of purified water. The wet granules are dried in a fluid bed dryer and passed through a screen. The colloidal silicon dioxide and the rest of the croscarmellose sodium are mixed, passed through a screen and blended with the dried granules in a V-blender. The magnesium stearate is passed through a screen, blended with the blend from the V-blender and afterwards the total mixture is compressed to tablets. The opadry yellow is suspended in purified water and the tablets are coated with the coating suspension.

### EXAMPLES13-15:

| Component | 60 mg | 120 mg | 180 mg |
|---|---|---|---|
| Starlix DS (H-form crystal modification) | 60 | 120 | 180 |
| Lactose Monohydrate | 141.5 | 283 | 214 |
| MicrocrystallineCellulose | 71 | 142 | 107 |
| Povidone K30 | 12 | 24 | 23 |
| Croscarmellose Sodium | 12 | 24 | 34 |
| Sub-Total (Granulation) | 296.5 | 593 | 558 |
| Croscarmellose Sodium | 6.4 | 12.8 | 24.5 |
| Colloidal Silicone Dioxide | 6.4 | 12.8 | 12.3 |
| Magnesium Stearate | 5.7 | 11.4 | 15.2 |
| Sub-Total (Core) | (315) | (630) | (610) |
| Opadry | 9 | 18 | 18 |
| Total | 324 | 648 | 628 |

What is claimed is:

1. A combination of at least two components selected from the group consisting of:
(i) a HMG CoA reductase inhibitor or a pharmaceutically acceptable salt thereof, selected from the group consisting of atorvastatin, cerivastatin, fluvastatin, lovastatin, pitavastatin, pravastatin, rosuvastatin, and simvastatin, and
(ii) a) an insulin secretion enhancer or a pharmaceutically acceptable salt thereof, or
b) an insulin sensitizer or a pharmaceutically acceptable salt thereof.

2. A combination of at least two components selected from the group consisting of:
(i) a HMG CoA reductase inhibitor or a pharmaceutically acceptable salt thereof, and
(ii) a) an insulin secretion enhancer or a pharmaceutically acceptable salt thereof, selected from the group consisting of :tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, a DPP-IV inhibitor, GLP1, GLP-1(7-36);Gln.sup.9-GLP-1(7-37); D -Gln.sup.9-GLP-1(7-37); acetyl-Lys.sup.9-GLP-1(7-37); Thr.sup.16-Lys.sup.18-GLP-1(7-37); and Lys.sup.18-GLP-1(7-37) or
b) an insulin sensitizer or a pharmaceutically acceptable salt thereof.

3. A combination according to claim 1 wherein the HMG CoA reductase inhibitor or a pharmaceutically acceptable salt thereof is selected from the group consisting of atorvastatin, fluvastatin, pitavastatin, and simvastatin.

4. A combination according to claim 1 wherein the HMG CoA reductase inhibitor or a pharmaceutically acceptable salt thereof is selected from the group consisting of fluvastatin, pitavastatin, and simvastatin.

5. A combination according to claim 1 wherein the HMG CoA reductase inhibitor or a pharmaceutically acceptable salt thereof is selected from the group consisting of fluvastatin, pitavastatin.

6. A combination according to claim 1 wherein the insulin secretion enhancer or a pharmaceutically acceptable salt thereof is selected from the group consisting of sulfonylureas (SU), glinides, DPP-IV inhibitors, GLP1 and GLP1 agonists.

7. A combination according to claim 1 wherein the insulin secretion enhancer or a pharmaceutically acceptable salt thereof is selected from the group consisting of, tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, a DPP-IV inhibitor, GLP1, GLP-1(7-36);Gln.sup.9-GLP-1 (7-37); D-Gln.sup.9-GLP-1 (7-37); acetyl-Lys.sup.9-GLP-1 (7-37); Thr.sup.16-Lys.sup.18-GLP-1(7-37); and Lys.sup.18-GLP-1(7-37).

8. A combination according to claim 1 wherein the insulin secretion enhancer or a pharmaceutically acceptable salt thereof is selected from the group consisting of, nateglinide and repaglinide.

9. A combination according to claim 1 wherein the insulin secretion enhancer is nateglinide or a pharmaceutically acceptable salt thereof.

10. A combination according to claim 1 wherein
a) the insulin secretion enhancer or a pharmaceutically acceptable salt thereof is nateglinide or a pharmaceutically acceptable salt thereof, or
b) the insulin secretion sensitizer metformin.

11. A combination according to claim 1 wherein the insulin secretion enhancer is pyrrolidine, 1-[(3-hydroxy-1-adamantyl)amino]acetyl-2-cyano-, (S), or a pharmaceutically acceptable salt thereof.

12. A combination according to claim 1 wherein the insulin secretion enhancer is 2-((5-cyanopyridin-2-yl)amino) ethyl or a pharmaceutically acceptable salt thereof.

13. A combination according to claim 1 wherein the insulin secretion enhancer is the compound 3-(4-(2-(2,3-Dihydro-1,4-benzothiazin-4-yl) ethoxy) phenyl)-2-ethoxypropanoic acid.

14. A combination according to claims 1 wherein the combination is a pharmaceutical combination.

15. A combination according to claim 1 for use in the prevention of, delay of progression of, treatment of a disease or condition selected from the group consisting of hyperlipidaemia, dyslipidemia, atherosclerosis, insulin resistance and syndrome X, diabetes mellitus type 2, obesity, nephropathy, renal failure, hypothyroidism, survival post myocardial infarction (Ml), coronary heart diseases, hypertension in the elderly, familial dyslipidemic hypertension, and remodeling following hypertension.

16. A method for the prevention, delay of progression or treatment of a disease and disorder which may be inhibited by the inhibition of HMG-Co-A reductase and/or by the enhancement of insulin secretion comprising administering to a warm-blooded animal, including man, in need thereof jointly therapeutically effective amounts of the composition comprising at least two therapeutic components selected from the group consisting of:
(i) a HMG CoA reductase inhibitor or a pharmaceutically acceptable salt thereof, selected from the group consisting of atorvastatin, cerivastatin, fluvastatin, lovastatin, pitavastatin, pravastatin, rosuvastatin, and simvastatin, and
(ii) a) an insulin secretion enhancer or a pharmaceutically acceptable salt thereof, or
b) an insulin sensitizer or a pharmaceutically acceptable salt thereof.

17. A method for the prevention, delay of progression or treatment of a disease and disorder which may be inhibited by the inhibition of HMG-Co-A reductase and/or by the enhancement of insulin secretion comprising administering to a warm-blooded animal, including man, in need thereof jointly therapeutically effective amounts of the composition comprising at least two therapeutic components selected from the group consisting of:
(i) a HMG CoA reductase inhibitor or a pharmaceutically acceptable salt thereof, and
(ii) a) an insulin secretion enhancer or a pharmaceutically acceptable salt thereof, selected from the group consisting of: tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolylcyclamide, nateglinide, repaglinide, mitiglinide, glimepiride, a DPP-IV inhibitor, GLP1, GLP-1(7-36);Gln.sup.9-GLP-1(7-37); D -Gln.sup.9-GLP-1(7-37); acetyl-Lys.sup.9-GLP-1(7-37); Thr.sup.16-Lys.sup.18-GLP-1(7-37); and Lys.sup.18-GLP-1(7-37) or
b) an insulin sensitizer or a pharmaceutically acceptable salt thereof.
